(19) United States
(12) Patent Application Publication
(10) Pub. No.: US 2025/0191723 A1
(43) Pub. Date: Jun. 12, 2025

(54) SYSTEM AND METHOD FOR MANAGING FITNESS VIA A PLURALITY OF DISPLAY WINDOWS

(71) Applicant: ShareSafe Solutions, LLC, Mobile, AL (US)

(72) Inventor: Robert B. Hanson, II, Mobile, AL (US)

(73) Assignee: ShareSafe Solutions, LLC, Mobile, AL (US)

(21) Appl. No.: 18/979,147

(22) Filed: Dec. 12, 2024

Related U.S. Application Data
(60) Provisional application No. 63/608,183, filed on Dec. 8, 2023.

(51) Int. Cl. G16H 20/30

(52) U.S. Cl. CPC G16H 20/30

(57) ABSTRACT

A system and method for managing fitness is provided. Generally, the system is designed to identify users of the system and manage fitness of the users using fitness devices and/or machine learning techniques. The system comprises a computing device having a user interface, at least one fitness device having at least one sensor configured to measure fitness data of a user, display operably connected to said computing device and said fitness device, processor operably connected to said computing device, fitness device, and display, and non-transitory computer-readable medium coupled to said processor and having instructions stored thereon.

### CROSS REFERENCES

This application claims priority to U.S. Provisional Application Ser. No. 63/608,183, filed on Dec. 8, 2023, in which application is incorporated herein in its entirety by reference.

### FIELD OF THE DISCLOSURE

The subject matter of the present disclosure refers generally to a system and method for managing fitness via computing devices, fitness devices, and displays.

### BACKGROUND

Physical fitness and exercise have become increasingly important aspects of modern life, with many individuals seeking to maintain their health through regular workout routines. While traditional gyms offer a variety of equipment and professional guidance, many people prefer the convenience and privacy of exercising at home. However, home fitness solutions often lack the comprehensive monitoring, feedback, and engagement features that make commercial gym experiences effective and motivating. Furthermore, inexpert individuals unsure of the most effective exercise programs are more likely to risk injury, purchase exercise equipment they do not know how to use or make limited progress by personally defined metrics of success. Such discouraging experiences are commonly cited reasons for discontinuing exercise, resulting in long-term declines in fitness and overall health.

Home fitness programs typically rely on a single display or screen such as a cell phone to provide workout instructions and tracking capabilities. This limited visual interface can make it difficult for users to simultaneously monitor multiple aspects of their workout, such as proper form, exercise instructions, heart rate, and other vital statistics. Additionally, users may struggle to maintain proper form and technique without real-time feedback, potentially leading to reduced effectiveness of their workouts or even injury. The emergence of connected fitness devices has introduced new possibilities for tracking and analyzing workout data. However, many existing systems fail to effectively integrate and present this information in a way that is both comprehensive and easily digestible during exercise. Users often must switch between different applications or devices to access various aspects of their fitness data, creating a disjointed and potentially distracting experience that can interrupt the flow of their workout.

While some fitness systems incorporate basic tracking and monitoring capabilities, they frequently lack sophisticated analysis tools that could provide deeper insights into user performance and progress. Traditional fitness tracking methods may collect data but often fall short in delivering personalized recommendations or adapting workout programs based on individual user patterns and needs. This limitation can result in generic fitness programs that fail to optimize results for any specific user. The increasing availability of fitness data and monitoring capabilities has created a need for more sophisticated systems that can effectively manage and present this information while providing intelligent analysis and feedback. Commercially available solutions typically do not fully leverage available technology to create comprehensive, adaptive fitness experiences that can rival the benefits of working with personal trainers in traditional gym settings. Furthermore, existing systems often struggle to maintain user engagement over time, as they lack the dynamic and responsive elements that could help sustain long-term motivation and commitment to fitness goals.

Accordingly, there is a need in the art for a system and method for presenting fitness data and content on a display having multiple display windows in a way that enhances a user's fitness experience.

### SUMMARY

A system and method for managing fitness is provided. In one aspect, the present invention is a system for linking and managing one or more displays. In another aspect, the present invention is a system for recording and storing user data, particularly fitness data. In yet another aspect, the present invention is a system which utilizes machine learning techniques to analyze user data stored therein, particularly fitness data, and generate predictions and recommendations to improve fitness outcomes. In still another aspect, the present invention is a method for utilizing the aforementioned system and analyzing its recorded data to generate predictions and recommendations to improve fitness outcomes. Generally, the present invention is a system and method for managing fitness by the coordination of displays, fitness devices, and computing devices with machine learning techniques.

The system comprises a computing device having a user interface, at least one fitness device having at least one sensor configured to measure fitness data of a user, and a display operably connected to the computing device and the at least one fitness device. The system further comprises a processor operably connected to the computing device, at least one fitness device, and display, and a non-transitory computer-readable medium coupled to the processor. A display user interface having a plurality of display windows may be used to present data on the display, and a control board operably connected to the display may configured to control the plurality of display windows presented within the display user interface. The control board may be configured to receive instructions from an input device operably connected to the processor, wherein the instructions cause the control board to change a layout of the plurality of display windows of the display user interface. The system may further comprise a secondary security device configured to verify an identity of the user.

According to another aspect of the present disclosure, a method for managing fitness is provided. The method includes receiving fitness data from at least one fitness device, wherein the fitness data comprises at least one sensor configured to measure the fitness data of a user, coupling the fitness data with a health plan of the user, wherein the health plan comprises a fitness plan, determining, via a machine learning technique, a fitness level of the user using the fitness data and the health plan, adjusting, via the machine learning technique, the fitness plan based on the fitness level to create an adjusted health plan, presenting the fitness data in a first display window of a plurality of display windows of a display user interface, and presenting the adjusted health plan in a second display window of the plurality of display windows. The health plan may further comprise a diet plan, and a computing device may be used to transmit nutrition data relevant to the health plan to the processor when input by the user. The method may further comprise additional operations, including: receiving the nutrition data from the computing device, determining, via the machine learning technique, a nutrition level of the user using the nutrition data and the nutrition plan, and adjusting, via the machine learning technique, the health plan based on the nutrition level to create the adjusted health plan.

The foregoing summary has outlined some features of the system and method of the present disclosure so that those skilled in the pertinent art may better understand the detailed description that follows. Additional features that form the subject of the claims will be described hereinafter. Those skilled in the pertinent art should appreciate that they can readily utilize these features for designing or modifying other structures for carrying out the same purpose of the system and method disclosed herein. Those skilled in the pertinent art should also realize that such equivalent designs or modifications do not depart from the scope of the system and method of the present disclosure.

### DESCRIPTION OF THE DRAWINGS

These and other features, aspects, and advantages of the present disclosure will become better understood with regard to the following description, appended claims, and accompanying drawings where:

FIG. 1 illustrates a system embodying features consistent with the principles of the present disclosure.

FIG. 2 illustrates a system embodying features consistent with the principles of the present disclosure.

FIG. 3 illustrates a system embodying features consistent with the principles of the present disclosure.

FIG. 4 illustrates a system embodying features consistent with the principles of the present disclosure.

FIG. 5 illustrates a system embodying features consistent with the principles of the present disclosure.

FIG. 6 illustrates a system embodying features consistent with the principles of the present disclosure.

FIG. 7 illustrates a system embodying features consistent with the principles of the present disclosure.

FIG. 8 illustrates the manner in which individual access to data may be granted or limited based on user roles and administrator roles.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For instance, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises”, and grammatical equivalents thereof are used herein to mean that other components, steps, etc. are optionally present. For instance, a system “comprising” components A, B, and C can contain only components A, B, and C, or can contain not only components A, B, and C, but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility). As will be evident from the disclosure provided below, the present invention satisfies the need for a system and method capable of managing fitness of its users.

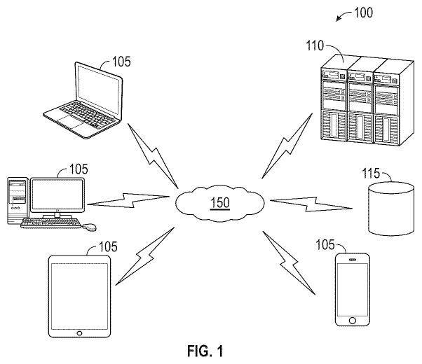

FIG. 1 depicts an exemplary environment 100 of the system 400 consisting of clients 105 connected to a server 110 and/or database 115 via a network 150. Clients 105 are devices of users 405 that may be used to access servers 110 and/or databases 115 through a network 150. A network 150 may comprise of one or more networks of any kind, including, but not limited to, a local area network (LAN), a wide area network (WAN), metropolitan area networks (MAN), a telephone network, such as the Public Switched Telephone Network (PSTN), an intranet, the Internet, a memory device, another type of network, or a combination of networks. In a preferred embodiment, computing entities 200 may act as clients 105 for a user 405. For instance, a client 105 may include a personal computer, a wireless telephone, a streaming device, a “smart” television, a personal digital assistant (PDA), a laptop, a smart phone, a tablet computer, or another type of computation or communication interface 280. Servers 110 may include devices that access, fetch, aggregate, process, search, provide, and/or maintain documents. Although FIG. 1 depicts a preferred embodiment of an environment 100 for the system 400, in other implementations, the environment 100 may contain fewer components, different components, differently arranged components, and/or additional components than those depicted in FIG. 1. Alternatively, or additionally, one or more components of the environment 100 may perform one or more other tasks described as being performed by one or more other components of the environment 100.

As depicted in FIG. 1, one embodiment of the system 400 may comprise a server 110. Although shown as a single server 110 in FIG. 1, a server 110 may, in some implementations, be implemented as multiple devices interlinked together via the network 150, wherein the devices may be distributed over a large geographic area and performing different functions or similar functions. For instance, two or more servers 110 may be implemented to work as a single server 110 performing the same tasks. Alternatively, one server 110 may perform the functions of multiple servers 110. For instance, a single server 110 may perform the tasks of a web server and an indexing server 110. Additionally, it is understood that multiple servers 110 may be used to operably connect the processor 220 to the database 115 and/or other content repositories. The processor 220 may be operably connected to the server 110 via wired or wireless connection. Types of servers 110 that may be used by the system 400 include, but are not limited to, search servers, document indexing servers, and web servers, or any combination thereof.

Search servers may include one or more computing entities 200 designed to implement a search engine, such as a documents/records search engine, general webpage search engine, etc. Search servers may, for instance, include one or more web servers designed to receive search queries and/or inputs from users 405, search one or more databases 115 in response to the search queries and/or inputs, and provide documents or information, relevant to the search queries and/or inputs, to users 405. In some implementations, search servers may include a web search server that may provide webpages to users 405, wherein a provided webpage may include a reference to a web server at which the desired information and/or links are located. The references to the web server at which the desired information is located may be included in a frame and/or text box, or as a link to the desired information/document. Document indexing servers may include one or more devices designed to index documents available through networks 150. Document indexing servers may access other servers 110, such as web servers that host content, to index the content. In some implementations, document indexing servers may index documents/records stored by other servers 110 connected to the network 150. Document indexing servers may, for instance, store and index content, information, and documents relating to user accounts and user-generated content. Web servers may include servers 110 that provide webpages to clients 105. For instance, the webpages may be HTML-based webpages. A web server may host one or more websites. As used herein, a website may refer to a collection of related webpages. Frequently, a website may be associated with a single domain name, although some websites may potentially encompass more than one domain name. The concepts described herein may be applied on a per-website basis. Alternatively, in some implementations, the concepts described herein may be applied on a per-webpage basis.

As used herein, a database 115 refers to a set of related data and the way it is organized. Access to this data is usually provided by a database management system (DBMS) consisting of an integrated set of computer software that allows users 405 to interact with one or more databases 115 and provides access to all of the data contained in the database 115. The DBMS provides various functions that allow entry, storage and retrieval of large quantities of information and provides ways to manage how that information is organized. Because of the close relationship between the database 115 and the DBMS, as used herein, the term database 115 refers to both a database 115 and DBMS.

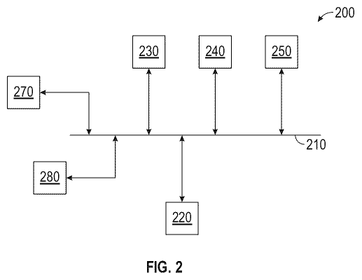

FIG. 2 is an exemplary diagram of a client 105, server 110, and/or or database 115 (hereinafter collectively referred to as “computing entity 200”), which may correspond to one or more of the clients 105, servers 110, and databases 115 according to an implementation consistent with the principles of the invention as described herein. The computing entity 200 may comprise a bus 210, a processor 220, memory 304, a storage device 250, a peripheral device 270, and a communication interface 280 (such as wired or wireless communication device). The bus 210 may be defined as one or more conductors that permit communication among the components of the computing entity 200. The processor 220 may be defined as logic circuitry that responds to and processes the basic instructions that drive the computing entity 200. Memory 304 may be defined as the integrated circuitry that stores information for immediate use in a computing entity 200. A peripheral device 270 may be defined as any hardware used by a user 405 and/or the computing entity 200 to facilitate communicate between the two. A storage device 250 may be defined as a device used to provide mass storage to a computing entity 200. A communication interface 280 may be defined as any transceiver-like device that enables the computing entity 200 to communicate with other devices and/or computing entities 200.

The bus 210 may comprise a high-speed interface 308 and/or a low-speed interface 312 that connects the various components together in a way such they may communicate with one another. A high-speed interface 308 manages bandwidth-intensive operations for computing device 300, while a low-speed interface 312 manages lower bandwidth-intensive operations. In some preferred embodiments, the high-speed interface 308 of a bus 210 may be coupled to the memory 304, display 316, and to high-speed expansion ports 310, which may accept various expansion cards such as a graphics processing unit (GPU). In other preferred embodiments, the low-speed interface 312 of a bus 210 may be coupled to a storage device 250 and low-speed expansion ports 314. The low-speed expansion ports 314 may include various communication ports, such as USB, Bluetooth, Ethernet, wireless Ethernet, etc. Additionally, the low-speed expansion ports 314 may be coupled to one or more peripheral devices 270, such as a keyboard, pointing device, scanner, and/or a networking device, wherein the low-speed expansion ports 314 facilitate the transfer of input data from the peripheral devices 270 to the processor 220 via the low-speed interface 312.

The processor 220 may comprise any type of conventional processor or microprocessor that interprets and executes computer readable instructions. The processor 220 is configured to perform the operations disclosed herein based on instructions stored within the system 400. The processor 220 may process instructions for execution within the computing entity 200, including instructions stored in memory 304 or on a storage device 250, to display graphical information for a graphical user interface (GUI) on an external peripheral device 270, such as a display 316. The processor 220 may provide for coordination of the other components of a computing entity 200, such as control of user interfaces 411, 511, 711, applications run by a computing entity 200, and wireless communication by a communication interface 280 of the computing entity 200. The processor 220 may be any processor or microprocessor suitable for executing instructions. In some embodiments, the processor 220 may have a memory device therein or coupled thereto suitable for storing the data, content, or other information or material disclosed herein. In some instances, the processor 220 may be a component of a larger computing entity 200. A computing entity 200 that may house the processor 220 therein may include, but are not limited to, laptops, desktops, workstations, personal digital assistants, servers 110, mainframes, cellular telephones, tablet computers, smart televisions, streaming devices, or any other similar device. Accordingly, the inventive subject matter disclosed herein, in full or in part, may be implemented or utilized in devices including, but are not limited to, laptops, desktops, workstations, personal digital assistants, servers 110, mainframes, cellular telephones, tablet computers, smart televisions, streaming devices, or any other similar device.

Memory 304 stores information within the computing device 300. In some preferred embodiments, memory 304 may include one or more volatile memory units. In another preferred embodiment, memory 304 may include one or more non-volatile memory units. Memory 304 may also include another form of computer-readable medium, such as a magnetic, solid state, or optical disk. For instance, a portion of a magnetic hard drive may be partitioned as a dynamic scratch space to allow for temporary storage of information that may be used by the processor 220 when faster types of memory, such as random-access memory (RAM), are in high demand. A computer-readable medium may refer to a non-transitory computer-readable memory device. A memory device may refer to storage space within a single storage device 250 or spread across multiple storage devices 250. The memory 304 may comprise main memory 230 and/or read only memory (ROM) 240. In a preferred embodiment, the main memory 230 may comprise RAM or another type of dynamic storage device 250 that stores information and instructions for execution by the processor 220. ROM 240 may comprise a conventional ROM device or another type of static storage device 250 that stores static information and instructions for use by processor 220. The storage device 250 may comprise a magnetic and/or optical recording medium and its corresponding drive.

As mentioned earlier, a peripheral device 270 is a device that facilitates communication between a user 405 and the processor 220. The peripheral device 270 may include, but is not limited to, an input device and/or an output device. As used herein, an input device may be defined as a device that allows a user 405 to input data and instructions that is then converted into a pattern of electrical signals in binary code that are comprehensible to a computing entity 200. An input device of the peripheral device 270 may include one or more conventional devices that permit a user 405 to input information into the computing entity 200, such as a controller, scanner, phone, camera, scanning device, keyboard, a mouse, a pen, voice recognition and/or biometric mechanisms, etc. As used herein, an output device may be defined as a device that translates the electronic signals received from a computing entity 200 into a form intelligible to the user 405. An output device of the peripheral device 270 may include one or more conventional devices that output information to a user 405, including a display 316, a printer, a speaker, an alarm, a projector, etc. Additionally, storage devices 250, such as CD-ROM drives, and other computing entities 200 may act as a peripheral device 270 that may act independently from the operably connected computing entity 200. For instance, a streaming device may transfer data to a smartphone, wherein the smartphone may use that data in a manner separate from the streaming device.

The storage device 250 is capable of providing the computing entity 200 mass storage. In some embodiments, the storage device 250 may comprise a computer-readable medium such as the memory 304, storage device 250, or memory 304 on the processor 220. A computer-readable medium may be defined as one or more physical or logical memory devices and/or carrier waves. Devices that may act as a computer readable medium include, but are not limited to, a hard disk device, optical disk device, tape device, flash memory or other similar solid-state memory device, or an array of devices, including devices in a storage area network or other configurations. Examples of computer-readable mediums include, but are not limited to, magnetic media such as hard disks, floppy disks, and magnetic tape; optical media such as CD ROM discs and DVDs; magneto-optical media such as optical discs; and hardware devices that are specially configured to store and perform programming instructions, such as ROM 240, RAM, flash memory, and the like.

In an embodiment, a computer program may be tangibly embodied in the storage device 250. The computer program may contain instructions that, when executed by the processor 220, performs one or more steps that comprise a method, such as those methods described herein. The instructions within a computer program may be carried to the processor 220 via the bus 210. Alternatively, the computer program may be carried to a computer-readable medium, wherein the information may then be accessed from the computer-readable medium by the processor 220 via the bus 210 as needed. In a preferred embodiment, the software instructions may be read into memory 304 from another computer-readable medium, such as data storage device 250, or from another device via the communication interface 280. Alternatively, hardwired circuitry may be used in place of or in combination with software instructions to implement processes consistent with the principles as described herein. Thus, implementations consistent with the invention as described herein are not limited to any specific combination of hardware circuitry and software.

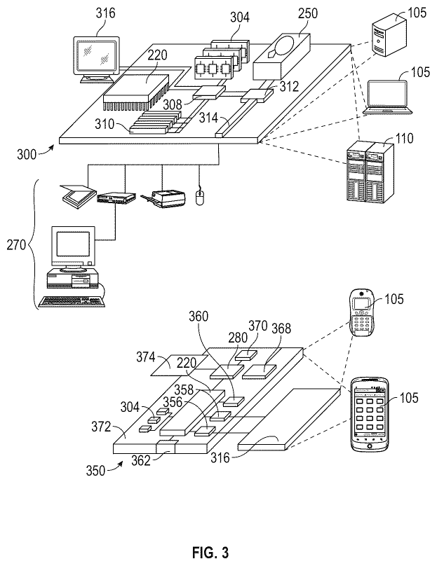

FIG. 3 depicts exemplary computing entities 200 in the form of a computing device 300 and mobile computing device 350, which may be used to carry out the various embodiments of the invention as described herein. A computing device 300 is intended to represent various forms of digital computers, such as laptops, desktops, workstations, servers 110, databases 115, mainframes, and other appropriate computers. A mobile computing device 350 is intended to represent various forms of mobile devices, such as scanners, scanning devices, personal digital assistants, cellular telephones, smart phones, tablet computers, and other similar devices. The various components depicted in FIG. 3, as well as their connections, relationships, and functions are meant to be examples only, and are not meant to limit the implementations of the invention as described herein. The computing device 300 may be implemented in a number of different forms, as shown in FIGS. 1 and 3. For instance, a computing device 300 may be implemented as a server 110 or in a group of servers 110. Computing devices 300 may also be implemented as part of a rack server system. In addition, a computing device 300 may be implemented as a personal computer, such as a desktop computer or laptop computer. Alternatively, components from a computing device 300 may be combined with other components in a mobile device, thus creating a mobile computing device 350. Each mobile computing device 350 may contain one or more computing devices 300 and mobile devices, and an entire system may be made up of multiple computing devices 300 and mobile devices communicating with each other as depicted by the mobile computing device 350 in FIG. 3. The computing entities 200 consistent with the principles of the invention as disclosed herein may perform certain receiving, communicating, generating, output providing, correlating, and storing operations as needed to perform the various methods as described in greater detail below.

In the embodiment depicted in FIG. 3, a computing device 300 may include a processor 220, memory 304 a storage device 250, high-speed expansion ports 310, low-speed expansion ports 314, and bus 210 operably connecting the processor 220, memory 304, storage device 250, high-speed expansion ports 310, and low-speed expansion ports 314. In one preferred embodiment, the bus 210 may comprise a high-speed interface 308 connecting the processor 220 to the memory 304 and high-speed expansion ports 310 as well as a low-speed interface 312 connecting to the low-speed expansion ports 314 and the storage device 250. Because each of the components are interconnected using the bus 210, they may be mounted on a common motherboard as depicted in FIG. 3 or in other manners as appropriate. The processor 220 may process instructions for execution within the computing device 300, including instructions stored in memory 304 or on the storage device 250. Processing these instructions may cause the computing device 300 to display graphical information for a GUI on an output device, such as a display 316 coupled to the high-speed interface 308. In other implementations, multiple processors and/or multiple buses may be used, as appropriate, along with multiple memory units and/or multiple types of memory. Additionally, multiple computing devices may be connected, wherein each device provides portions of the necessary operations.

A mobile computing device 350 may include a processor 220, memory 304 a peripheral device 270 (such as a display 316, a communication interface 280, and a transceiver 368, among other components). A mobile computing device 350 may also be provided with a storage device 250, such as a micro-drive or other previously mentioned storage device 250, to provide additional storage. Preferably, each of the components of the mobile computing device 350 are interconnected using a bus 210, which may allow several of the components of the mobile computing device 350 to be mounted on a common motherboard as depicted in FIG. 3 or in other manners as appropriate. In some implementations, a computer program may be tangibly embodied in an information carrier. The computer program may contain instructions that, when executed by the processor 220, perform one or more methods, such as those described herein. The information carrier is preferably a computer-readable medium, such as memory, expansion memory 374, or memory 304 on the processor 220 such as ROM 240, that may be received via the transceiver or external interface 362. The mobile computing device 350 may be implemented in a number of different forms, as shown in FIG. 3. For instance, a mobile computing device 350 may be implemented as a cellular telephone, part of a smart phone, personal digital assistant, or other similar mobile device.

The processor 220 may execute instructions within the mobile computing device 350, including instructions stored in the memory 304 and/or storage device 250. The processor 220 may be implemented as a chipset of chips that may include separate and multiple analog and/or digital processors. The processor 220 may provide for coordination of the other components of the mobile computing device 350, such as control of the user interfaces 411, 511, 711, applications run by the mobile computing device 350, and wireless communication by the mobile computing device 350. The processor 220 of the mobile computing device 350 may communicate with a user 405 through the control interface 358 coupled to a peripheral device 270 and the display interface 356 coupled to a display 316. The display 316 of the mobile computing device 350 may include, but is not limited to, Liquid Crystal Display (LCD), Light Emitting Diode (LED) display, Organic Light Emitting Diode (OLED) display, and Plasma Display Panel (PDP), holographic displays, augmented reality displays, virtual reality displays, or any combination thereof. The display interface 356 may include appropriate circuitry for causing the display 316 to present graphical and other information to a user 405. The control interface 358 may receive commands from a user 405 via a peripheral device 270 and convert the commands into a computer readable signal for the processor 220. In addition, an external interface 362 may be provided in communication with processor 220, which may enable near area communication of the mobile computing device 350 with other devices. The external interface 362 may provide for wired communications in some implementations or wireless communication in other implementations. In a preferred embodiment, multiple interfaces may be used in a single mobile computing device 350 as is depicted in FIG. 3.

Memory 304 stores information within the mobile computing device 350. Devices that may act as memory 304 for the mobile computing device 350 include, but are not limited to computer-readable media, volatile memory, and non-volatile memory. Expansion memory 374 may also be provided and connected to the mobile computing device 350 through an expansion interface 372, which may include a Single In-Line Memory Module (SIM) card interface or micro secure digital (Micro-SD) card interface. Expansion memory 374 may include, but is not limited to, various types of flash memory and non-volatile random-access memory (NVRAM). Such expansion memory 374 may provide extra storage space for the mobile computing device 350. In addition, expansion memory 374 may store computer programs or other information that may be used by the mobile computing device 350. For instance, expansion memory 374 may have instructions stored thereon that, when carried out by the processor 220, cause the mobile computing device 350 perform the methods described herein. Further, expansion memory 374 may have secure information stored thereon; therefore, expansion memory 374 may be provided as a security module for a mobile computing device 350, wherein the security module may be programmed with instructions that permit secure use of a mobile computing device 350. In addition, expansion memory 374 having secure applications and secure information stored thereon may allow a user 405 to place identifying information on the expansion memory 374 via the mobile computing device 350 in a non-hackable manner.

A mobile computing device 350 may communicate wirelessly through the communication interface 280, which may include digital signal processing circuitry where necessary. The communication interface 280 may provide for communications under various modes or protocols, including, but not limited to, Global System Mobile Communication (GSM), Short Message Services (SMS), Enterprise Messaging System (EMS), Multimedia Messaging Service (MMS), Code Division Multiple Access (CDMA), Time Division Multiple Access (TDMA), Personal Digital Cellular (PDC), Wideband Code Division Multiple Access (WCDMA), IMT Multi-Carrier (CDMAX 0), and General Packet Radio Service (GPRS), or any combination thereof. Such communication may occur, for example, through a transceiver 368. Short-range communication may occur, such as using a Bluetooth, WIFI, or other such transceiver 368. In addition, a Global Positioning System (GPS) receiver module 370 may provide additional navigation- and location-related wireless data to the mobile computing device 350, which may be used as appropriate by applications running on the mobile computing device 350. Alternatively, the mobile computing device 350 may communicate audibly using an audio codec 360, which may receive spoken information from a user 405 and covert the received spoken information into a digital form that may be processed by the processor 220. The audio codec 360 may likewise generate audible sound for a user 405, such as through a speaker, e.g., in a handset of mobile computing device 350. Such sound may include sound from voice telephone calls, recorded sound such as voice messages, music files, etc. Sound may also include sound generated by applications operating on the mobile computing device 350.

The system 400 may comprise a power supply, which may be any source of power that provides the system 400 with the required energy. In a preferred embodiment, the power supply may be a stationary power source that has been installed in a way such that it is fastened in place, such as a 3-prong wall outlet. In a preferred embodiment, the stationary power source is connected to the wiring system of a premises. In another preferred embodiment, the power supply may be a mobile power source, such as a battery pack. In a preferred embodiment, mobile power source does not need to be connected to the wiring system of a premises to provide power to the system but may be capable of connecting to the wiring system of said premises to provide power to a system connected thereto. In another preferred embodiment, the system 400 may comprise multiple power supplies configured to supply power to the system 400 in different circumstances. For instance, the system 400 may be directly plugged into a stationary power source, which may provide power to the system 400 so long as the system does not move out of range of said stationary power source, as well as connected to a mobile power source, which may provide power to the system 400 when the system 400 is not connected to a stationary power source or in situations where the stationary power source ceases to provide power to the system 400.

The system 400 may comprise a power supply, which may be any source of power that provides the system 400 with the required energy. In a preferred embodiment, the power supply may be a stationary power source that has been installed in a way such that it is fastened in place, such as a 3-prong wall outlet. In a preferred embodiment, the stationary power source is connected to the wiring system of a premises, such as a house or a building. In another preferred embodiment, the power supply may be a mobile power source, such as a battery pack, gas-powered generator, and fuel cell. In a preferred embodiment, the mobile power source does not need to be connected to the wiring system of a premises to provide power to the system but may be capable of connecting to the wiring system of said premises to provide power to a system connected thereto. In another preferred embodiment, the system 400 may comprise multiple power supplies configured to supply power to the system 400 in different circumstances. For instance, the system 400 may be directly plugged into a stationary power source, which may provide power to the system 400 so long as the system does not move out of range of said stationary power source, as well as connected to a mobile power source, which may provide power to the system 400 when the system 400 is not connected to a stationary power source or in situations where the stationary power source ceases to provide power to the system 400. In yet another preferred embodiment, a plurality of solar charging panels may be operably connected to a battery of the system, which may then supply power to the system either directly or via the wiring of the premises. As such, the system 400 may be configured to receive power in a variety of ways without departing from the inventive subject matter described herein.

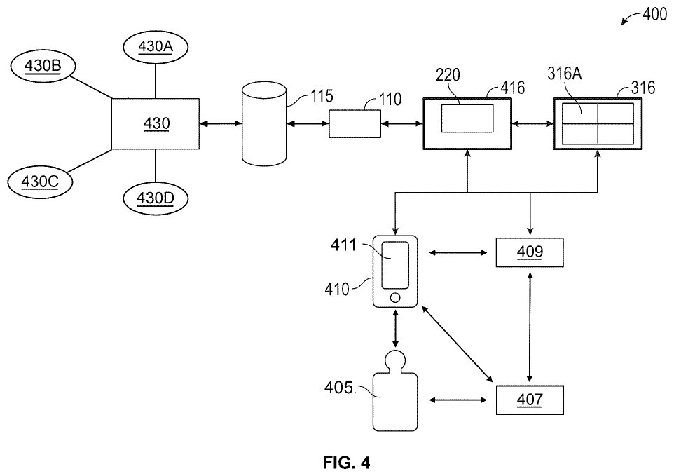

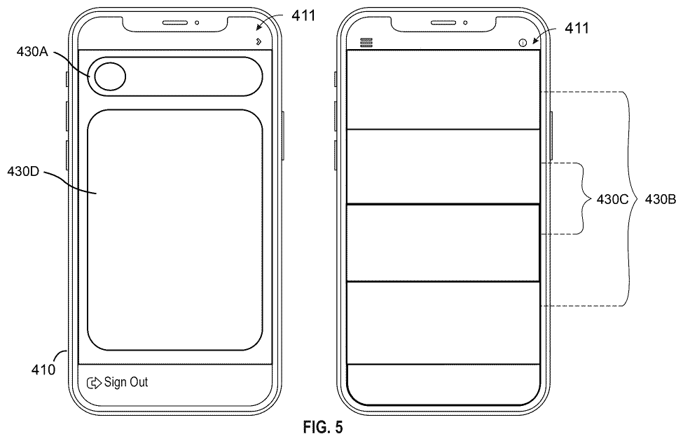

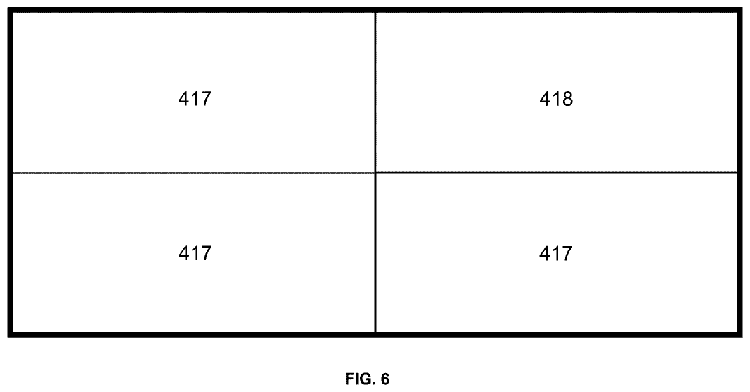

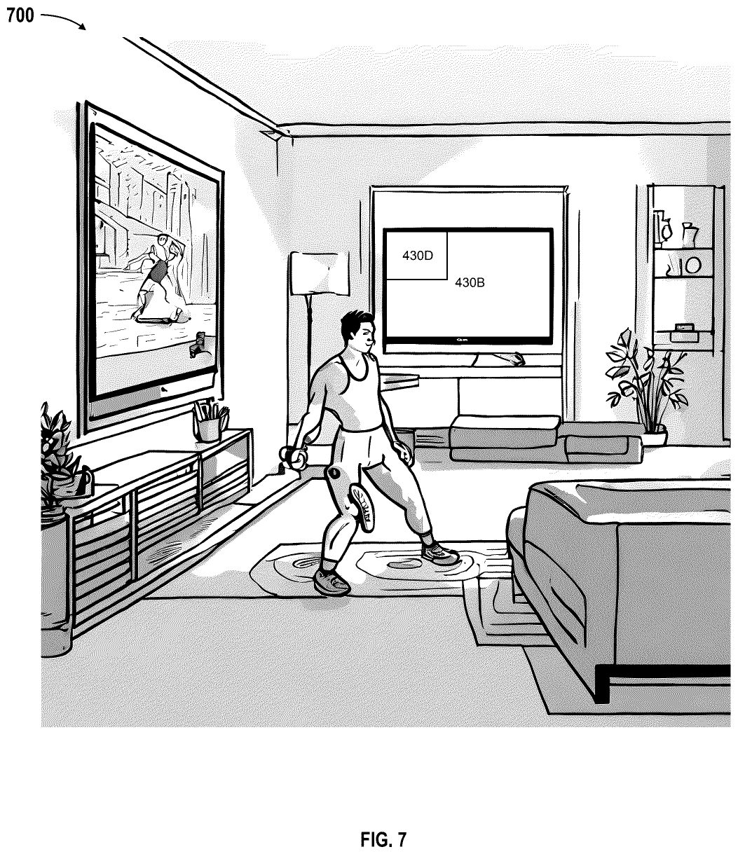

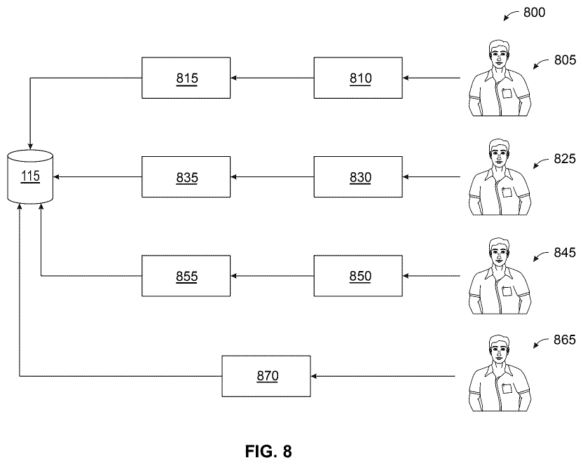

FIGS. 4-8 illustrate embodiments of a system 400 and methods for managing fitness via a display device having a display user interface and operably connected to one or more computing devices and fitness devices of a user. FIG. 4 illustrates a preferred embodiment of the system 400 having a computing device 410, display 316, at least one fitness device, and a processor operably connected to said computing device, display, and at least one fitness device. FIG. 5 illustrates an example user interface 411 of the computing device 410, wherein a display 316 operably connected to said computing device 410 may split the display user interface into multiple windows containing user data 430A, image data 430B, application data 430C, and fitness data 430D. FIG. 6 illustrate an example display user interface 316A having a plurality of display windows showing user data 430A, image data 430B, application data 430C, and fitness data 430D pertaining to the fitness of a user, wherein a control board 409 operably connected to said display 316 may receive a computer readable signal from the computing device 410 containing said user data 430A, image data 430B, application data 430C, and fitness data 430D. FIG. 7 illustrates the system 400 being used by a user 405 within an environment 700 to manage fitness of a user. FIG. 8 illustrates permission levels 800 that may be utilized by the present system 400 for controlling access to user content such as user data 430A, image data 430B, application data 430C, and fitness data 430D. It is understood that the various method steps associated with the methods of the present disclosure may be carried out as operations by the system 400 shown in FIGS. 4-7.

The system 400 generally comprises a computing device 410 having a user interface 411, at least one fitness device having at least one sensor configured to measure fitness data of a user, display operably connected to said computing device and said fitness device, processor 220 operably connected to said computing device, fitness device, and display, and non-transitory computer-readable medium (CRM) 416 coupled to said processor 220 and having instructions stored thereon. Some preferred embodiments may further comprise a camera 905 operably connected to computing devices 410, displays 316, and/or secondary security devices. In one preferred embodiment, a database 115 may be operably connected to the processor 220 and the various data of the system 400 may be stored therein, including, but not limited to, user data 430A, image data 430B, application data 430C, and fitness data 430D. In some preferred embodiments, the displays 316 may further comprise a display user interface 316A having a plurality of display windows configured to present the various data of the system 400 therein, wherein control boards 409 of the displays 316 may be configured to receive the various data of the system and arrange the plurality of display windows within the display user interface. In yet another preferred embodiment, a wireless communication interface may allow the processors 220 of the system 400 to receive and transmit the various data of the system therebetween.

Though some embodiments may mention a single computing device 410 of a user 405, one with skill in the art will recognize that multiple computing devices 410 of multiple users may be used without departing from the inventive subject matter described herein. Additionally, though some embodiments may refer to a single display, one with skill in the art will recognize that multiple displays may be linked together in a way that creates a “single” display that may be used in a manner not departing from the inventive subject matter described herein. For instance, four OLED televisions may be linked together in way that creates a multi-display that the system may use as a “single” display. Additionally, one with skill in the art will recognize that a plurality of displays may be controlled by a single control board, and the single control board may manage the plurality of display windows about the display user interfaces of the plurality of displays. In yet another preferred embodiment, two or more control boards of two or more displays may be operably connected to one another and manage the plurality of display windows about the display user interfaces of the plurality of displays in collaboration with one another. Accordingly, one with skill in the art will recognize that displays may be used in combination with one or more control boards and one or more computing devices in a number of ways without departing from the inventive subject matter described herein.

Generally, the system is designed to identify users of the system and manage fitness of the users. Users may operably connect to display devices via computing devices and select data to be presented within a display user interface of the display. A user may manipulate the user interface of the computing device in a way that allows said user to choose a fitness instructional video to be presented on the display in the form of image data. In some preferred embodiments, a user 405 may be required to use a secondary security method to access a display to access the various features of the system, including access to fitness instructors, medical professionals, fitness plans, diet plans, and fitness instructional videos. For instance, a user 405 may be required to use a camera of their computing device 410 to scan a predefined pattern, such as a bar code or a QR code, that is presented on a display 316, which may associate that user with a particular display and allow them to access features of the display thanks to permission levels associated within their user profile. In some preferred embodiments, a secondary security method may also serve as a means as determining a location of a user by using display data of the display to relate the location of the display to the system.

Fitness instructional videos may be defined as image data in the form of a pre-recorded exercise video and/or diet video designed to educate a user as to a manner in which said user can improve their health through physical activity and/or dietary planning. For instance, a fitness instructional video may include image data showing a fitness instructor performing a plurality of exercises in order to encourage a user to perform said plurality of exercises in the same manner as the fitness instructor. In a preferred embodiment, fitness instructional videos of the system further comprise audio data related to the subject matter of the image data. For instance, the fitness instructional video may include music and motivational talking from a fitness instructor to encourage a user to push past a perceived physical limit, such as an exercise weight maximum and/or exercise repetition maximum. In another preferred embodiment, a user may choose a fitness instructional video in the form of a live feed, wherein the live feed comprises image data collected by one or more cameras connected to a display device. For instance, a fitness instructor may use the system to create a live feed and allow other users to join, wherein the image data may be transmitted to the displays of the other users whereas fitness data of the other users may be transmitted to the display of the fitness instructor. In another preferred embodiment, a user may select a fitness instructor and/or healthcare professional, causing a camera of a fitness instructor and/or healthcare professional to transmit image data to the display of the user. Additionally, the system may transmit image data from a camera of the user to the display of the fitness instructor and/or healthcare professional.

In a preferred embodiment, a control board 409 of a display 316 receives user data 430A, image data 430B, application data 430C, and/or fitness data 430D from a computing entity 200 and/or fitness device. The control board 409 may then present said user data 430A, image data 430B, application data 430C, and/or fitness data 430D via the display 316 in the display user interface 316A. In another preferred embodiment, the display may be configured to receive user data 430A, image data 430B, application data 430C, and/or fitness data 430D via a server and/or database when selected by a user via the user interface of the computing device and/or the display user interface of the display. In a preferred embodiment, image data 430B is streamed/mirrored from the computing entity 200, server 110, and/or database 115 to the control board 409, wherein the control board 409 inserts said streamed/mirrored image data 430B into the display user interface 316A. Alternatively, the control board 409 may manipulate image data 430B and/or the display user interface 316A based on commands received from an input device. In one preferred embodiment, the display user interface 316A may also comprise a control window, which may allow a user 405 to control the layout of the plurality of display windows of the display user interface 316A. For instance, a user 405 may choose a layout that separates the display user interface 316A into multiple windows arranged in a particular way. In some embodiments, the control window may allow a user to alter the size and orientation of a display window of the display user interface. Alternatively, an input device having a plurality of layouts thereon may be used to manipulate the layout of the display user interface 316A. The input device may be connected to the system 400 via a wired or wireless connection. In a preferred embodiment, the input device transmits a computer readable signal containing instructions to the control board 409, which the control board 409 uses to manipulate data presented via the display user interface 316A.

In a preferred embodiment, a user 405 logs into a user profile of the system before accessing the various features of a display, allowing the system to verify the identity of the user. A user interface 411 of a computing device 410 allows a user to input login credentials and/or commands. A processor 220 operably connected to said computing device and said display 316 sends the login credentials and/or commands to a control board of said display via a computer readable signal, wherein said login credentials and/or commands of said computer readable signal allow access to said display should they be associated with a user profile having sufficient permission levels. A user may then manipulate the user interface of the computing device in a way that allows said user to choose various data of the system to be presented on the display for review. In some preferred embodiments, a user 405 may be required to use a secondary security method to access a display to present the various data of the system. For instance, a user 405 may be required to use a camera of their computing device 410 to scan a predefined pattern, such as a bar code or a QR code, that is presented on a display 316, which may associate that user with a particular display.

In a preferred embodiment, displays of the system are configured for remote communication. Preferably, a first user uses a secondary security method to link a first computing device to a first display and second user uses a secondary security method to link a second computing device to a second display. Once connected, the users may select the various data of the system which they would like to be presented within a display window of the displays. For instance, a user in the homeplace may use a secondary security method to associate a display within the homeplace with their computing device and user profile. One or more fitness instructors at a fitness facility may be logged into a second display of the system, allowing said one or more fitness instructors to select various data of the system to be presented within the display windows of the display user interface. The displays are preferably operably connected to one another in a way such that data presented within the display windows of the display is the same. However, though the same data may be presented within the display windows of operably connected displays, the display windows may or may not be organized in the same manner within the display user interfaces of the displays. In a preferred embodiment, each control board of a display controls how the content is organized within display windows of the display user interface.

A fitness device may be defined as a device configured to measure physical activity of a user. Fitness devices 407 that may be operably connected to the computing device and/or display include, but are not limited to, heart rate monitors, accelerometers, global positioning systems (GPS), galvanic skin response sensors, thermometers, ambient light sensors, UV sensors, or any combination thereof. Fitness devices may also be connected to an apparatus or machine designed to facilitate one or more types of exercise, such as a leg press. Such devices may record repetitions, time between sets, resistance, and other data as fitness data 430D associated with the user profile 430, which may then be used as training data for AI analytics. In one preferred embodiment, fitness devices 407 are moveably attached to a display, allowing a user to remove the fitness device and attach it to their person so that said fitness device may collect fitness data for the system. For instance, a heart rate monitor may be removably secured to the display in a way such that a user 405 may remove said heart rate monitor from a mount of said display so that hear rate data may be collected and saved in the form of fitness data 430B. In another preferred embodiment, fitness devices 407 may be configured to operably connect to the display and not be physically secured to the display in any way. For instance, a user's smart watch-having an accelerometer and gyroscope configured to collect fitness data 430B in the form of linear acceleration data and angular acceleration data, respectively—may be used by the system to collect fitness data during a fitness session, wherein the fitness data is presented in one or more display windows of the display user interface along with image data of a fitness instructional video.

In some preferred embodiments, the system 400 may further comprise a secondary security device. Devices that may act as the secondary security device may include, but are not limited to, biometric devices, key cards, wearables, or any combination thereof. In a preferred embodiment, devices that may act as biometric devices include, but are not limited to, contact biometric devices, such as fingerprint scanners and hand geometry scanners, and/or non-contact biometric devices, such as face scanners, iris scanners, retina scanners, palm vein scanners, and voice identification devices. In some embodiments, the secondary security device may be operably connected to the computing device 410 and/or display 316 in a way such that it is in direct communication with the computing device 410 and/or display 316 and no other computing device 410 and/or display 316. For instance, a secondary security device in the form of a facial recognition camera may be securely and directly connected to a control board 409 of the display 316 such that a user 405 must biometrically scan their face prior to the system allowing access to the various data of the system. In some preferred embodiments, biometric data associated with a user is saved in a user profile as user data, which the system uses to verify a user's identity. For instance, secondary security devices may be securely and directly connected to a first computing device and a second computing device in a way such that both a first user of the first computing device and a second user of the second computing device must biometrically scan thumbprints prior to the system allowing the first user and second user to access data of the system.

In a preferred embodiment, key cards and wearables preferably comprise a secure transmitter configured to transmit a login credentials to the computing device and/or control board of the display. Wearables having a secure transmitter include clothing and accessories, such as shirts, pants, jackets, belts, shoes, wristbands, watches, glasses, pins, nametags, etc., that have said transmitter attached thereto and/or incorporated therein. The secure transmitter preferably contains login credentials in the form of a unique ID, which may be conveyed to a computing device and/or control board of a display 316 in the form of a computer readable signal. Unique IDs contained within the computer readable signal that has been broadcast by the transmitter may include, but are not limited to, unique identifier codes, social security numbers, personal identification numbers (PINs), etc. For instance, a computer readable signal broadcast by a secondary security device in the form of a wrist band may contain information that will alert the control board of the display 316 that a particular user 405 is within a certain range, which may cause the system 400 to allow a user to access data of the system if additional steps are taken.

Types of devices that may act as the transmitter include, but are not limited, to near field communication (NFC), Bluetooth, infrared (IR), radio-frequency communication (RFC), radio-frequency identification (RFID), and ANT+, or any combination thereof. In an embodiment, transmitters may broadcast signals of more than one type. For instance, a transmitter comprising an IR transmitter and RFID transmitter may broadcast IR signals and RFID signals. Alternatively, a transmitter may broadcast signals of only one type of signal. For instance, identification (ID) cards may be fitted with transmitters that broadcast NFC signals containing unique IDs associated with a particular user, wherein displays equipped with NFC receivers must receive said NFC signals containing unique IDs before access to one or more features of the display user interface may be granted.

Use of secondary security devices may be used solely or in addition to secondary security methods of the system, allowing the system to have flexible multifactor identification. Simultaneous use may be beneficial to prevent unauthorized access to data of the system and/or communications between users of the system, including personal trainers and/or healthcare professionals. For instance, a user may use both a secondary security method and biometric scanner for identification purposes before allowing a user to access the various features of the system. In another preferred embodiment, the system may use a secondary security method for identification purposes and a wearable for activating other features of the system, such as access to paid fitness classes, personal trainers, and/or healthcare professionals. For instance, a user may use a secondary security method to allow the system to identify a user and associate a computing device of the user with a display. The secure transmitter of a wearable in the form of a smartwatch may transmit a computer readable signal to the display in a way such that it will allow a user to access additional features of the system that allow access to fitness instructors and dieticians. Fitness devices of the user may transmit fitness data to the display where it may be presented to fitness instructors during a fitness instructional video.

In a preferred embodiment, the various data of the system 400 may be stored in user profiles 430. In a preferred embodiment, a user profile 430 is related to a particular user 405. A user 405 is preferably associated with a particular user profile 430 based on a username. However, it is understood that a user 405 may be associated with a user profile 430 using a variety of methods without departing from the inventive subject matter described herein. Types of data that may be stored within user profiles 430 of the system 400 include, but are not limited to, user data 430A, image data 430B, application data 430C, and fitness data 430D. Some preferred embodiments of the system 400 may comprise a database 115 operably connected to the processor 220. The database 115 may be configured to store user data 430A, image data 430B, application data 430C, and fitness data 430D within user profiles 430 and/or separately. As used herein, user data 430A may be defined as personal information of a user 405 that helps the system 400 identify the user 405 and their interests. Types of data that may be used by the system 400 as user data 430A includes, but is not limited to, a user's name, username, social security number, phone number, email address, physical address, gender, age, or any combination thereof.

As used herein, image data 430B may be defined as photographic or trace objects that represent the underlying pixel data of an area of an image element, which is created, collected, and stored using image constructor devices, such as a camera. This data may then be used by the system in the various manners as described herein. For instance, the system may use image data obtained via a scanning device and/or a secondary security device to confirm the identity of a user. For instance, image data of a fitness instructional video may be transmitted to the display and presented to the user. For instance, image data of an application may be transmitted to the display from the computing device, server, and/or database where it may be manipulated by the control board.

Application data may be defined as instructions that cause a display application of the display to perform an action. In one preferred embodiment, the system may determine whether a user application of the computing device is compatible with a display application of the display. If it is determined that the display application and user application are compatible, application data may be transmitted to the display from the computing device in lieu of image data. The display application is controlled by the control board of the display and inserted into a display window of the display user interface. Instructions input into a compatible user application are transmitted to the control board from the computing device and are used by the control board to perform actions of the display application, reducing the amount of data transferred between the computing device and display. For instance, a fitness device operably connected to the computing device may transmit fitness data to a user application version of a fitness application of the computing device. A display application version of the fitness application and the user application version of said fitness application may be compatible in a way such that a user may open the user application version on their computing device and subsequently instruct the system (via the user interface) to display the user application version in a display window of the display user interface. The processor of the control board may then determine if the display application version of the fitness application is compatible with the user application version of the fitness application. If the display application version and user application version are compatible, the control board may open the display application version of the fitness application locally and manipulate it via instructions received from the computing device as fitness data is received or actions are taken via the user application version. If the display application version and user application version are not compatible, the control board may receive image data of the user application version and present it within a display window of the display user interface.

As previously mentioned, some preferred embodiments of the display 316 may further comprise a control board 409. The control board 409 comprises at least one circuit and microchip. In another preferred embodiment, the control board 409 may further comprise a wireless communication interface, which may allow the control board 409 to receive instructions from an input device controlled by a user 405. In a preferred embodiment, the control board 409 may control the plurality of display windows of the display user interface 316A. The microchip of the control board 409 comprises a microprocessor and memory. In another preferred embodiment, the microchip may further comprise a wireless communication interface in the form of an antenna. The microprocessor may be defined as a multipurpose, clock driven, register based, digital-integrated circuit which accepts binary data as input, processes it according to instructions stored in its memory, and provides results as output. In a preferred embodiment, the microprocessor may receive the various data of the system from a server 110 and/or database 115 via the wireless communication interface.

As mentioned previously, the system 400 may comprise a user interface 411. A user interface 411 may be defined as a space where interactions between a user 405 and the system 400 may take place. In an embodiment, the interactions may take place in a way such that a user 405 may control the operations of the system 400. A user interface 411 may include, but is not limited to operating systems, command line user interfaces, conversational interfaces, web-based user interfaces, zooming user interfaces, touch screens, task-based user interfaces, touch user interfaces, text-based user interfaces, intelligent user interfaces, brain-computer interfaces (BCIs), and graphical user interfaces, or any combination thereof. The system 400 may present data of the user interface 411 to the user 405 via a display 316 operably connected to the processor 220. A display 316 may be defined as an output device that communicates data that may include, but is not limited to, visual, auditory, cutaneous, kinesthetic, olfactory, and gustatory, or any combination thereof.

In some preferred embodiments, the user interface and/or display user interface may comprise additional controls that allow users of the system to manipulate how the various data of the system is presented within the display windows. In a preferred embodiment, access to these features is based on permission levels of the user. For instance, the system may be configured in a way such that certain fitness instructional videos are only accessible should the user have the appropriate permissions. For instance, the system may be configured in a way such that a user may only access dietary features of the display user interface should the user have a permission level that grants access to said dietary feature. In one preferred embodiment, users may only select data to be presented within display windows of the system should the user have appropriate permission levels.

In a preferred embodiment, the control board 409 of the display 316 receives image data 430B from the computing device, server 110, and/or database 115 and may then present said image data 430B via at least one display window of the display user interface 316A of a display 316, as illustrated in FIGS. 4-7. In a preferred embodiment, image data is streamed/mirrored from the computing device, database 115, and/or server 110 to the control board 409, wherein the control board 409 inserts said streamed/mirrored image data 430B into said at least one display window. Alternatively, the control board 409 may automatically select a layout of the display user interface 316A, wherein said layout may be determined based on a plurality of variables, including, but not limited to, number of users, type of content being viewed by the user(s) 405, user preferences, user location, or any combination thereof. For instance, the control board 409 may select a layout of a display user interface 316A comprising a split screen having two display windows configured to present a workout checklist in a first display window and image data of a fitness instructional video in a second display window. For instance, the control board 409 may select a layout of a display user interface 316A comprising a split screen having two display windows and communication window, wherein a fitness instructor is presented in the communication window, wherein image data showing how a workout should be performed is presented in the first display window, wherein image data pertaining to a number of repetitions a user has and/or is required to perform is the second display window.

Information presented via a display 316 may be referred to as a soft copy of the information because the information exists electronically and is presented for a temporary period of time. Information stored on the non-transitory computer-readable medium 416 may be referred to as the hard copy of the information. For instance, a display 316 may present a soft copy of visual information via a liquid crystal display (LCD), wherein the hardcopy of the visual information is stored on a local hard drive. For instance, a display 316 may present a soft copy of audio information via a speaker, wherein the hard copy of the audio information is stored in RAM. For instance, a display 316 may present a soft copy of tactile information via a haptic suit, wherein the hard copy of the tactile information is stored within a database 115. Displays 316 may include, but are not limited to, cathode ray tube monitors, LCD monitors, light emitting diode (LED) monitors, gas plasma monitors, screen readers, speech synthesizers, haptic feedback equipment, virtual reality headsets, speakers, and scent generating devices, or any combination thereof.

The database 115 may be operably connected to the processor 220 via wired or wireless connection. In a preferred embodiment, the database 115 is configured to store user data 430A, image data 430B, application data 430C, and fitness data 430D within user profiles 430. Alternatively, the user data 430A, image data 430B, application data 430C, and fitness data 430D may be stored within user profiles 430 on the non-transitory computer-readable medium 416. The database 115 may be a relational database such that the user data 430A, image data 430B, application data 430C, and fitness data 430D associated with each user profile 430 within the plurality of user profiles 430 may be stored, at least in part, in one or more tables. Alternatively, the database 115 may be an object database such that user data 430A, image data 430B, application data 430C, and fitness data 430D associated with each user profile 430 of the plurality of user profiles 430 may be stored, at least in part, as objects. In some instances, the database 115 may comprise a relational and/or object database and a server 110 dedicated solely to managing the user data 430A, image data 430B, application data 430C, and fitness data 430D in the manners disclosed herein.

In a preferred embodiment, the system 400 may use artificial intelligence (AI) techniques to perform functions of the system. In one preferred embodiment, AI techniques may be used to control the number of display windows presented within the display user interface. In yet another preferred embodiment, AI techniques may be used to organize the plurality of display windows within the display user interface. In another preferred embodiment, AI techniques may be used to evaluate fitness data of a user to determine a fitness level of a user. In another preferred embodiment, AI techniques may be used to evaluate fitness data and alter a fitness program of the user based on said fitness data. In yet another preferred embodiment, AI techniques may be used to evaluate fitness data pertaining to a user's diet so that the system may alter a user's diet. The term “artificial intelligence” and grammatical equivalents thereof are used herein to mean an intelligence method used by the system 400 to correctly interpret and learn from data of the system 400 or a plurality of systems in order to achieve specific goals and tasks through flexible adaptation. Types of intelligence methods that may be used by the system 400 include, but are not limited to, machine learning, neural network, computer vision, or any combination thereof.

In a preferred embodiment, the system 400 uses AI techniques to perform in-depth recommendations and calibrations to the fitness program of a user 405. For instance, machine learning techniques could train an AI to associate exercises and stretches with specific muscle groups. An AI trained with such a data set could predict the occurrence of muscle strain in specific muscle groups based on the parameters of a workout program and suggest appropriate stretches and cooldown routines to mitigate pain and stiffness. In another preferred embodiment, the AI is trained using a data set comprising different workout regimens associated with specific sports or activities, certain body forms, certain muscle groups of emphasis, or other parameters. The AI so trained can thereby generate novel fitness plans and regimens for a user 405 based on the user's stated fitness goals and limitations. For instance, a user 405 with no access to home gymnasium equipment like weights might specify “bodyweight” to ensure highly relevant workout regimens. In yet another preferred embodiment, deep learning language models are used to train the AI to associate patterns of fitness or exercise with key phrases not directly connected to technical descriptions of muscle groups. For instance, a user 405 interested in a “superhero” build might be provided a fitness program emphasizing high-intensity weightlifting that builds muscle quickly. Alternatively, a user desiring a “swimmer's build” but without access to a body of water could be given a fitness program designed to reduce adipose and strengthen the arms, shoulders, back, and chest. In still another preferred embodiment, AI techniques could be used to make recommendations about exercise equipment that the user 405 would find useful based on their prior fitness programs. For instance, a user that exclusively used yoga-based fitness programs might be recommended yoga mats or resistance bands.

The system 400 preferably uses machine learning techniques to perform the methods disclosed herein, wherein the instructions carried out by the processor 220 for said machine learning techniques are stored on the non-transitory computer-readable medium 416, server 110, and/or database 115. Machine learning techniques that may be used by the system 400 include, but are not limited to, classification algorithms, neural network algorithm, regression algorithms, decision tree algorithms, clustering algorithms, genetic algorithms, supervised learning algorithms, semi-supervised learning algorithms, unsupervised learning algorithms, deep learning algorithms, or other types of algorithms. More specifically, machine learning algorithms can include implementations of one or more of the following algorithms: support vector machine, decision tree, nearest neighbor algorithm, random forest, ridge regression, Lasso algorithm, k-means clustering algorithm, boosting algorithm, spectral clustering algorithm, mean shift clustering algorithm, non-negative matrix factorization algorithm, elastic net algorithm, Bayesian classifier algorithm, RANSAC algorithm, orthogonal matching pursuit algorithm, bootstrap aggregating, temporal difference learning, backpropagation, online machine learning, Q-learning, stochastic gradient descent, least squares regression, logistic regression, ordinary least squares regression (OLSR), linear regression, stepwise regression, multivariate adaptive regression splines (MARS), locally estimated scatterplot smoothing (LOESS) ensemble methods, clustering algorithms, centroid based algorithms, principal component analysis (PCA), singular value decomposition, independent component analysis, k nearest neighbors (kNN), learning vector quantization (LVQ), self-organizing map (SOM), locally weighted learning (LWL), apriori algorithms, eclat algorithms, regularization algorithms, ridge regression, least absolute shrinkage and selection operator (LASSO), elastic net, classification and regression tree (CART), iterative dichotomiser 3 (ID3), C4.5 and C5.0, chi-squared automatic interaction detection (CHAID), decision stump, M5, conditional decision trees, least-angle regression (LARS), naive bayes, gaussian naïve bayes, multinomial naïve bayes, averaged one-dependence estimators (AODE), bayesian belief network (BBN), bayesian network (BN), k-medians, expectation maximisation (EM), hierarchical clustering, perceptron back-propagation, hopfield network, radial basis function network (RBFN), deep boltzmann machine (DBM), deep belief networks (DBN), convolutional neural network (CNN), stacked auto-encoders, principal component regression (PCR), partial least squares regression (PLSR), sammon mapping, multidimensional scaling (MDS), projection pursuit, linear discriminant analysis (LDA), mixture discriminant analysis (MDA), quadratic discriminant analysis (QDA), flexible discriminant analysis (FDA), bootstrapped aggregation (bagging), adaboost, stacked generalization (blending), gradient boosting machines (GBM), gradient boosted regression trees (GBRT), random forest, or even algorithms yet to be invented.

In a preferred embodiment, the system may determine a fitness level of a user based on fitness data collected by the system. For instance, the system may be operably connected to a fitness device configured to measure heart rate of a user. Based on the user's heart rate and the workout plan followed by a user, the system may use unsupervised learning to make adjustment to the intensity of the workout plan. In a preferred embodiment, the system may make dietary recommendations to a user based on fitness data to help a user reach a fitness goal. For instance, the system may collect fitness data from a weight scale that informs the system as to the weight of the user. Based on fitness data obtained by the system in the form of the user's dietary intake, the system may adjust a user's diet to assist a user to reach a weight goal as set by a user. In some preferred embodiments, fitness data collected and processed by the system may be monitored using a machine learning technique to determine when a user may be at risk of injury due to declining athletic performance. For instance, fitness data in the form of linear acceleration data and angular acceleration data may be used to determine when a user is becoming physically exhausted after performing repetitive exercises, wherein the system may recommend that a user take a break to recover in order to prevent injury through overuse.

In a preferred embodiment, the system 400 may use more than one machine learning technique to monitor fitness data of a user. For instance, the system 400 comprising a microphone may use a combination of NLP and reinforcement learning to discern food items that a user has consumed in a day as well as determine which macro/micro nutrients the user still should consume. In another preferred embodiment, the system 400 may actively monitor a fitness data to determine if a user may be experiencing a potential medical emergency. For instance, the system 400 comprising a heart rate monitor and camera may use a combination of facial emotion recognition (FER) and deep learning to discern if a user is experiencing any irregular heart activity during exercise. Should the system determine that a user is experiencing irregular heart activity during exercise, the system may recommend that the user stop the exercise and consult a medical professional, which in some embodiments may be facilitated via a communication window of the system. In another preferred embodiment, the system may contact emergency medical services should the fitness data indicate that a user is experiencing a medical emergency.

In another preferred embodiment, the system 400 may use image data 430B in conjunction with machine learning techniques to analyze user 405 completion of a particular fitness program. For instance, a camera associated with a fitness device 407 might provide image data 430B to the AI while a user is completing an exercise. The AI might then use the image data to count and display the number of repetitions of the exercise. This count could then be compared with a given fitness plan to assess completion. In another preferred embodiment, the user provides image data 430B in the form of an image of their desired build. The AI could use this image data 430B to estimate adiposity and prominence of specified muscle groups in the depicted build and generate a fitness program targeting said muscle groups. In yet another preferred embodiment, the user 405 provides image data 430B in the form of their own build. The AI may then compare the user's build to the image of their desired build in order to estimate progress toward their desired build. In still another preferred embodiment, the image data 430B of the user 405 is used as part of a data set for machine learning techniques to assess the efficacy of different exercises on user fitness, adiposity, and muscle growth. When making recommendations for fitness program composition, more efficient exercises might be selected to improve user fitness.

In a preferred embodiment, the machine learning techniques comprise instructions configured to create a trained machine learning techniques from at least some training data and according to an implementation of the machine learning techniques, wherein the training data serves as a baseline dataset that may act as the foundational data of the machine learning techniques. The instructions of the machine learning techniques dictate how the machine learning techniques gain knowledge from the various data sources of the system and may comprise various types of programmable instructions that include, but are not limited to, local commands, remote commands, executable files, protocol commands, selected commands, or any combination thereof. The instructions of the machine learning techniques may vary widely, depending on a desired implementation. In a preferred embodiment, instructions may include streamed-lined instructions that instruct the machine learning techniques on how to train the system, possibly in the form of a script (e.g., Python, Ruby, JavaScript, etc.). In another preferred embodiment, the instructions may include data filters or data selection criteria that define requirements for desired results sets created from the various data of the system as well as which machine learning algorithm is to be used.

Training of the machine learning techniques may be supervised, semi-supervised, or unsupervised. In some preferred embodiments, the machine learning systems may use NLP to analyze data (e.g., audio data, text data, etc.). For instance, the system may use natural language processing and deep learning to ascertain a baseline voice of a user when not physically exhausted and when physically exhausted, which may be used by the system to determine when a user may be feeling physically exhausted. Training of the machine learning techniques may result in baseline machine learning techniques that may serve as AI techniques for performing the various functions of the system in the manners described herein. Baseline machine learning techniques may further be configured to act as passive models or active models. A passive model may be described as a final, completed machine learning model that uses only the baseline data set to establish behavior of the baseline machine learning technique. An active model may be described as a plasticity machine learning model that is dynamic in that it may be updated using both the baseline dataset and data outside of the baseline data set.

In a preferred embodiment, the system may use a passive model to allow for a high degree of control as to how the system manages user interfaces and display windows in the manners described herein. For instance, a passive model may be configured via a private dataset to provide each user of the system with the same dietary and exercise recommendations. These recommendations may be made by the system regardless of user data that may indicate that particular users have historically preferred other dietary and exercise recommendations. A passive model may be especially useful for users having user profiles with little user data from which the machine learning techniques may learn from. In some preferred embodiments, the system may be configured to begin as passive models until a threshold amount of user data has been acquired. Once the threshold amount of user data has been acquired, the system may cause the machine learning techniques to switch to active models, allowing the system to make recommendations to a user that better parallel historical preferences of the user. For instance, a system may be configured to make exercise recommendations to the user based on a passive model for the first 30 exercise recommendations, wherein the system may also be configured to determine if a user followed the exercise recommendations made by the system. After the system has made 30 exercise recommendations, the machine learning techniques of the system may switch to an active machine model for that particular user and make exercise recommendations based on the exercises chosen by the user after recommendations had been made by the system.

In some embodiments, an active machine model may be updated in real-time, daily, weekly, bimonthly, monthly, quarterly, or annually using the various data (e.g., to update model instructions, shifts in time, new/corrected private data sets, user data, fitness data, etc.), of the system. In some preferred embodiments, the passive machine model may also be updated as new/updated private data sets become available. In a preferred embodiment, machine learning techniques comprise metadata that describe the state of the passive/active model with respect to its updates. The metadata may include attributes describing one or more of the following: a version number, date updated, amount of new data used for the update, shifts in model parameters, convergence requirements, or other information. Because each user of the system may potentially have a unique machine learning technique associated with their user profile due to the personal nature of user data associated with each user profile, such information allows for identifying distinct passive/active models within the system that may be separately managed.

To prevent un-authorized users from accessing other user's information, the system 400 may employ a security method. As illustrated in FIG. 8, the security method of the system 400 may comprise a plurality of permission levels 800 that may grant users 405 access to user content 815, 835, 855 within the database while simultaneously denying users 405 without appropriate permission levels 800 the ability to view user content 815, 835, 855. To access the user content 815, 835, 855 stored within the database 115, users 405 may be required to make a request via a user interface 411. Access to the data within the database 115 may be granted or denied by the processor 220 based on verification of a requesting user's 805, 825, 845 permission level 800. If the requesting user's 805, 825, 845 permission level 800 is sufficient, the processor 220 may provide the requesting user 805, 825, 845 access to user content 815, 835, 855 stored within the database. Conversely, if the requesting user's 805, 825, 845 permission level 800 is insufficient, the processor 220 may deny the requesting user 805, 825, 845 access to user content 815, 835, 855 stored within the database. In an embodiment, permission levels 800 may be based on user roles 810, 830, 850 and administrator roles 870, as illustrated in FIG. 8. User roles 810, 830, 850 allow requesting users 805, 825, 845 to access user content 815, 835, 855 that a user 405 has uploaded and/or otherwise obtained through use of the system 400. Administrator roles 870 allow administrators 865 to access system 400 wide data.

In an embodiment, user roles 810, 830, 850 may be assigned to a user 405 in a way such that a requesting user 805, 825, 845 may view user profiles 430 containing user data 430A, image data 430B, application data 430C, and fitness data 430D via a user interface 411. To access the data within the database 115, a user 405 may make a user request via the user interface 411 to the processor 220. In an embodiment, the processor 220 may grant or deny the request based on the permission level 800 associated with the requesting user 805, 825, 845. Only users 405 having appropriate user roles 810, 830, 850 or administrator roles 870 may access the data within the user profiles 430. For instance, as illustrated in FIG. 8, requesting user 1 805 has permission to view user 1 content 815 and user 2 content 835 whereas requesting user 2 825 only has permission to view user 2 content 835. Alternatively, user content 815, 835, 855 may be restricted in a way such that a user may only view a limited amount of user content 815, 835, 855. For instance, requesting user 3 845 may be granted a permission level 800 that only allows them to view user 3 content 855 related to their specific interest but not user 3 content 855 related to the identity of said user 405. In the example illustrated in FIG. 8, an administrator 865 may bestow a new permission level 800 on users 405 so that it may grant them greater permissions or lesser permissions. For instance, an administrator 865 may bestow a greater permission level 800 on other users 405 so that they may view user 3's content 855 and/or any other user's content 815, 835, 855. Therefore, the permission levels 800 of the system 400 may be assigned to users 405 in various ways without departing from the inventive subject matter described herein.

In a preferred embodiment, the system 400 is installed in a public or commercial gymnasium to assist the members of said gymnasium plan, manage, and track their progress towards their personal fitness goals. The gymnasium can thereby use the key features of the system 400 to manage the members' experience. For instance, the members of a gymnasium might be given access to a user interface 411 on their personal computing devices 410 in the form of a mobile phone-based application. These members may thereby create user profiles 430 to access the system's fitness data management functions, AI analytics, and display management on both their personal computing devices 410 and nearby displays 316. In a preferred embodiment, the gymnasium creates a plurality of permission levels 800 to manage user access to the system 400, which may be restricted by employment status or type of gymnasium membership. For instance, a member with the least expensive type of membership might be given access to only a set of prerecorded fitness videos. By contrast, a member with a membership of intermediate expense might be given access to AI-mediated fitness tracking and personalized workout recommendations based on stated fitness goals. A member with the most expensive membership might be further given access to AI-mediated dietary recommendations, workout form analytics, and workout planning to target particular body shapes and types. In a preferred embodiment, employees of the gymnasium have access to user-specific fitness data based on their duties and clientele. For example, a personal trainer might have a user profile 430 with access to fitness data 430D for specified other users in order to track a client's progress even when the trainer is not present to assist the client. However, the personal trainer might not be given access to the system 400 features for their own personal use.

The system 400 has a plurality of uses inside the home and in a commercial gymnasium; however, its applicability as a training and monitoring tool can be broadly useful for institutions that study or require regular exercise. In a preferred embodiment, the system 400 is installed in sporting facilities to help professional athletes to train and meet their fitness requirement in the off-season, pre-season, and season of their respective sport. For instance, a football coach might use the system 400 to monitor their players and ensure they are on track for the season. Furthermore, the details of the fitness data submitted to AI analytics could inform AI suggestions about relative player strengths and rates of recovery from game-induced injuries, informing position selections. In universities and similar institutions studying exercise science, physiology, and public health install the system 400 in their facilities for the benefit of both their research and their students, staff, and faculty. The system's capacity for recording and analyzing fitness data 430D, both associated with a user profile 430 and anonymized, is an invaluable research tool for scientists and analysts interested in population-wide and individual initiatives involving exercise and fitness. In yet another preferred embodiment, the system 400 is used at hospitals, in occupational therapy and physical therapy outpatient clinics, and by medical professionals visiting a patient user at home. Recovering from surgery, illness, or injury with the aid of physical or occupational therapy is an extended process that can require carefully planned, monitored, regular exercise. By monitoring a patient user 405 and tracking their progress, the system 400 can facilitate a therapist's duties, allowing them to attend more patients with greater efficiency.

In another preferred embodiment, military training is supplemented during boot camp, officer training, and routine fitness checks by the system 400. A fort or other military might automate routine fitness tests by referring to fitness data 430D associated with a given user 405, reducing time-consuming testing for personnel. In yet another preferred embodiment, the system 400 is installed in an emergency response center like a firehouse to facilitate fitness training and testing for emergency responders such as firefighters. Similar to military organizations, emergency responders often have stringent fitness requirements; unlike military organizations, emergency response organizations are frequently leanly funded and staffed by volunteers. For such organizations, a collective commercial gym membership could swiftly prove an onerous financial burden. An AI-enhanced fitness aid such as the aforementioned system 400, including pair of display screens and home gymnasium equipment could easily equal the service provided by a commercial gymnasium at a fraction of the cost.

The subject matter described herein may be embodied in systems, apparati, methods, and/or articles depending on the desired configuration. In particular, various implementations of the subject matter described herein may be realized in digital electronic circuitry, integrated circuitry, specially designed application specific integrated circuits (ASICs), computer hardware, firmware, software, and/or combinations thereof. These various implementations may include implementation in one or more computer programs that may be executable and/or interpretable on a programmable system including at least one programmable processor, which may be special or general purpose, coupled to receive data and instructions from, and to transmit data and instructions to, a storage system, and at least one peripheral device.

These computer programs, which may also be referred to as programs, software, applications, software applications, components, or code, may include machine instructions for a programmable processor, and may be implemented in a high-level procedural and/or object-oriented programming language, and/or in assembly machine language. As used herein, the term “non-transitory computer-readable medium” refers to any computer program, product, apparatus, and/or device, such as magnetic discs, optical disks, memory, and Programmable Logic Devices (PLDs), used to provide machine instructions and/or data to a programmable processor, including a non-transitory computer-readable medium that receives machine instructions as a computer-readable signal. The term “computer-readable signal” refers to any signal used to provide machine instructions and/or data to a programmable processor. To provide for interaction with a user, the subject matter described herein may be implemented on a computer having a display device, such as a cathode ray tube (CRD), liquid crystal display (LCD), light emitting display (LED) monitor for displaying information to the user and a keyboard and a pointing device, such as a mouse or a trackball, by which the user may provide input to the computer. Displays may include, but are not limited to, visual, auditory, cutaneous, kinesthetic, olfactory, and gustatory displays, or any combination thereof.

Other kinds of devices may be used to facilitate interaction with a user as well. For instance, feedback provided to the user may be any form of sensory feedback, such as visual feedback, auditory feedback, or tactile feedback; and input from the user may be received in any form including, but not limited to, acoustic, speech, or tactile input. The subject matter described herein may be implemented in a computing system that includes a back-end component, such as a data server, or that includes a middleware component, such as an application server, or that includes a front-end component, such as a client computer having a graphical user interface or a Web browser through which a user may interact with the system described herein, or any combination of such back-end, middleware, or front-end components. The components of the system may be interconnected by any form or medium of digital data communication, such as a communication network. Examples of communication networks may include, but are not limited to, a local area network (“LAN”), a wide area network (“WAN”), metropolitan area networks (“MAN”), and the internet.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For instance, the implementations described above can be directed to various combinations and subcombinations of the disclosed features and/or combinations and subcombinations of several further features disclosed above. In addition, the logic flow depicted in the accompanying figures and/or described herein do not necessarily require the particular order shown, or sequential order, to achieve desirable results. It will be readily understood to those skilled in the art that various other changes in the details, devices, and arrangements of the parts and method stages which have been described and illustrated in order to explain the nature of this inventive subject matter can be made without departing from the principles and scope of the inventive subject matter.

What is claimed is:

1. A system for managing fitness, comprising:
a computing device having a user interface;
at least one fitness device having at least one sensor configured to measure fitness data of a user;
a display operably connected to said computing device and said at least one fitness device, wherein said display comprises a display user interface having a plurality of display windows;
a processor operably connected to said computing device, at least one fitness device, and display; and
a non-transitory computer-readable medium coupled to said processor,
wherein said non-transitory computer-readable medium contains instructions stored thereon, which, when executed by said processor, cause said processor to perform operations comprising:
receiving said fitness data from said at least one fitness device,
coupling said fitness data with a health plan of said user,
determining, via a machine learning technique, a fitness level of said user using said fitness data and said health plan,
adjusting, via said machine learning technique, said health plan based on said fitness level to create an adjusted health plan,
presenting said fitness data in a first display window of said plurality of display windows,
presenting said adjusted health plan in a second display window of said plurality of display windows.

2. The system of claim 1, wherein said fitness data comprises at least one of: heart rate data, linear acceleration data, angular acceleration data, and weight data.

3. The system of claim 1, wherein said health plan comprises at least one of a workout plan or a diet plan.

4. The system of claim 1, wherein said computing device transmits nutrition data relevant to said health plan to said processor when input by said user.

5. The system of claim 4, further comprising additional instructions, which, when executed by said processor, cause said processor to perform additional operations comprising:
receiving said nutrition data from said computing device,
determining, via said machine learning technique, a nutrition level of said user using said nutrition data and said health plan, and
adjusting, via said machine learning technique, said health plan based on said nutrition level to create said adjusted health plan.

6. The system of claim 1, wherein said display further comprises a control board configured to control said plurality of display windows presented within said display user interface.

7. The system of claim 4, wherein said control board is configured to receive instructions from an input device operably connected to said processor, wherein said instructions cause said control board to change a layout of said plurality of display windows of said display user interface.

8. The system of claim 1, wherein said system further comprises a secondary security device configured to verify an identity of said user.

9. The system of claim 6, wherein said secondary security device comprises at least one of: a biometric device, a key card, and a wearable device having a secure transmitter.

10. A method for managing fitness, comprising:
receiving fitness data from at least one fitness device,
wherein said fitness data comprises at least one sensor configured to measure said fitness data of a user,
coupling said fitness data with a health plan of said user,
wherein said health plan comprises a fitness plan,
determining, via a machine learning technique, a fitness level of said user using said fitness data and said health plan;
adjusting, via said machine learning technique, said fitness plan based on said fitness level to create an adjusted health plan;
presenting said fitness data in a first display window of a plurality of display windows of a display user interface; and
presenting said adjusted health plan in a second display window of said plurality of display windows.

11. The method of claim 10, wherein said fitness data comprises at least one of: heart rate data, linear acceleration data, angular acceleration data, and weight data.

12. The method of claim 10, wherein said health plan further comprises a diet plan.

13. The method of claim 12, wherein a computing device transmits nutrition data relevant to said health plan to said processor when input by said user,

14. The system of claim 13, further comprising additional instructions, which, when executed by said processor, cause said processor to perform additional operations comprising:
receiving said nutrition data from said computing device,
determining, via said machine learning technique, a nutrition level of said user using said nutrition data and said nutrition plan, and
adjusting, via said machine learning technique, said health plan based on said nutrition level to create said adjusted health plan.

15. The method of claim 10, further comprising:
controlling, via a control board of said display, said plurality of display windows presented within said display user interface.

16. The method of claim 15, further comprising:
receiving instructions from an input device operably connected to said processor; and
changing a layout of said plurality of display windows of said display user interface based on said instructions.

17. The method of claim 10, further comprising:
verifying an identity of said user via a secondary security device.

18. The method of claim 17, wherein said secondary security device comprises at least one of: a biometric device, a key card, and a wearable device having a secure transmitter.

19. A system for managing fitness, comprising:
a computing device having a user interface;
at least one fitness device having at least one sensor configured to measure fitness data of a user;
a display operably connected to said computing device and said at least one fitness device, wherein said display comprises a display user interface having a plurality of display windows;
a processor operably connected to said computing device, at least one fitness device, and display; and
a non-transitory computer-readable medium coupled to said processor,
wherein said non-transitory computer-readable medium contains instructions stored thereon, which, when executed by said processor, cause said processor to perform operations comprising:
receiving said fitness data from said at least one fitness device,
coupling said fitness data with a health plan of said user,
determining, via a machine learning technique, a fitness level of said user using said fitness data and said health plan,
adjusting, via said machine learning technique, said health plan based on said fitness level to create an adjusted health plan,
presenting said fitness data in a first display window of said plurality of display windows,
presenting said adjusted health plan in a second display window of said plurality of display windows,
receiving nutrition data from said computing device,
determining, via said machine learning technique, a nutrition level of said user using said nutrition data and said health plan, and
adjusting, via said machine learning technique, said health plan based on said nutrition level to create said adjusted health plan.

20. The system of claim 19, further comprising a control board operably connected to said display,
wherein said control board is configured to control said plurality of display windows presented within said display user interface,
wherein said control board is configured to receive instructions from an input device operably connected to said processor, wherein said instructions cause said control board to change a layout of said plurality of display windows of said display user interface.
